(19) United States
(12) Patent Application Publication
(10) Pub. No.: US 2007/0037208 A1
(43) Pub. Date: Feb. 15, 2007

(54) Method for detecting cardiac ischemia via changes in b-natriuretic peptide levels

(76) Inventors: Robert S. Foote, Hartland, VT (US); Kiang-Tech Jerry Yeo, Etna, NH (US)

Correspondence Address: LICATA & TYRRELL P.C., 66 E. MAIN STREET, MARLTON, NJ, 08053, US

(21) Appl. No.: 10/553,585

(22) PCT Filed: May 17, 2004
(86) PCT No.: PCT/US04/15341

§ 371 Date: Jan. 13, 2006

Related U.S. Application Data
(60) Provisional application No. 60/474,201, filed on May 29, 2003.
(60) Provisional application No. 60/501,494, filed on Sep. 9, 2003.

(51) Int. Cl. G01N 33/53; G01N 33/50

(52) U.S. Cl. 435/7.1; 436/86

(57) ABSTRACT

The present invention relates to a method of detecting cardiac ischemia by measuring the levels of BNP or NT-proBNP. Increases in BNP or NTproBNP levels in an individual are indicative of cardiac ischemia.

### BACKGROUND OF THE INVENTION

Exercise electrocardiography (EKG) is the most widely used noninvasive method to detect the presence of coronary artery disease (CAD). However, its usefulness is limited by relatively modest sensitivity and specificity (Gianrossi, et al. (1989) Circulation 80:87-98; Froelicher, et al. (1998) Ann. Intern. Med. 128:965-74; Morise and Diamond (1995) Am. Heart J. 130:741-7). In addition, the EKG cannot be interpreted in patients with left bundle branch block, left ventricular hypertrophy, digitalis therapy, pre-excitation, marked hypertension, or significant baseline ST abnormalities. Other more accurate nonivasive tests are available, e.g., exercise echocardiography and exercise testing with radionuclide imaging, but these are less widely available and considerably more expensive.

B-natriuretic peptide (BNP) is a neurohormone with diuretic, vasodilatory, and renin-angiotensin-aldosterone antagonistic effects. It is secreted by cells in the ventricular wall in response to increases in wall stress (Espiner, et al. (1995) Endocrinol. Metab. Clin. North Am. 24:481-509; Yasue, et al. (1994) Circulation 90:195-203; Mair, et al. (2001) Clin. Chem. Lab. Med. 39:571-88). The prohormone is cleaved to a smaller active form and a larger amino-terminal inactive form (NTproBNP)(Hunt, et al. (1997) Clin. Endocrinol. (Oxf) 47:287-96). Both of these peptides have been shown to have diagnostic or prognostic value in a variety of left and right ventricular structural and functional abnormalities, particularly heart failure (Espiner, et al. (1995) supra; Dao, et al. (2001) J. Am. Coll. Cardiol. 37:379-85; Davis, et al. (1994) Lancet 343:440-4), as well as in systolic (McDonagh, et al. (1998) Lancet 351:9-13; Talwar, et al. (2000) Br. J. Clin. Pharmacol. 50:15-20) and diastolic (Lang, et al. (1994) Am. Heart J. 127:1635-6; Lubien, et al. (2002) Circulation 105:595-601) dysfunction, unstable angina (Kikuta, et al. (1996) Am. Heart J. 132:101-7; Talwar, et al. (2000) Heart 84:421-4), acute coronary syndromes (de Lemos, et al. (2001) New Engl. J. Med. 345:1014-21; Jernberg, et al. (2002) J. Am. Coll. Cardiol. 40:437-45; Omland, et al. (2002) Am. J. Cardiol. 89:463-5) and myocardial infarction (Darbar, et al. (1996) Am. J. Cardiol. 78:284-7; Morita, et al. (1993) Circulation 88:82-91). In addition, two studies (Toth, et al. (1994) Am. J. Physiol. 266:H1572-80; Goetze, et al. (2003) FASEB J. 17:1105-7) have found evidence that tissue hypoxia alone may trigger release of BNP in the absence of left ventricular dysfunction.

Exercise-induced ischemia is known to produce wall-motion abnormalities in the affected area of the ventricle (Crouse, et al. (1991) Am. J. Cardiol. 67:1213-8). However, few studies have examined the effect of exercise on cardiac markers in plasma. Four studies (Kohno, et al. (1992) Clin. Exp. Pharmacol. Physiol. 19:193-200; Nicholson, et al. (1993) Clin. Exp. Pharmacol. Physiol. 20:535-40; Marumoto, et al. (1995) Japan. Circ. J. 59:715-24; Marumoto, et al. (1995) Clin. Sci. 88:551-6) examined the effect of single episodes of exercise on BNP levels; of these, two included patients with CAD and data on nuclear perfusion imaging (Marumoto, et al. (1995) supra; Marumoto, et al. (1995) supra). Although there was a trend toward increases in BNP in patients with CAD compared to normal controls, the studies were limited by small sample sizes, unmatched controls, submaximal work loads and peak heart rates, and a lack of documentation of ischemia. No studies have correlated BNP levels with ischemia in individual patients, and studies examining the effect of exercise on NTproBNP levels appear to be lacking. Although normal resting levels of BNP and NTproBNP are similar, evidence (Hunt, et al. (1997) supra; Richards, et al. (1998) Circulation 97:1921-9) suggests that in cardiac impairment the proportional and absolute increments above normal levels of NTproBNP exceed those of BNP.

A need exists for a more sensitive marker of early cardiac dysfunction. The present invention meets this long felt need by providing assays for measuring levels of BNP or NTproBNP which are indicative of cardiac ischemia.

### SUMMARY OF THE INVENTION

An object of the present invention is to provide a method for detecting cardiac ischemia in an individual. The method involves measuring the level of a natriuretic peptide in a sample isolated from an individual and comparing said level to a control, wherein an increase in the level in the sample as compared to the control is indicative of cardiac ischemia in the individual. In one preferred embodiment of the present invention, the natriuretic peptide is brain natriuretic peptide (BNP) or N-terminal probrain natriuretic peptide (NTproBNP), or a fragment thereof. In another preferred embodiment of the present invention the control is isolated from an individual before the individual has conducted an exercise test and the sample is isolated from the same individiual after the individual has conducted an exercise test.

### DETAILED DESCRIPTION OF THE INVENTION

It has now been shown that the level of a natriuretic peptide is useful in diagnosing cardiac ischemia. Results provided herein demonstrate that patients with cardiac ischemia have higher median levels of BNP or NTproBNP than patients without cardiac ischemia. It has further been shown that measurements of exercise-induced increases in these natriuretic peptides more than doubles the sensitivity of an exercise test in detecting cardiac ischemia with no loss of specificity.

The diagnostic sensitivity and specificity of measuring levels of BNP and NTproBNP were was determined in 74 individuals. Of 74 patients enrolled in exercise stress testing, 40 were classified as having perfusion defects on stress imaging that reversed at rest (ischemic group); 14 (35%) of these patients also had fixed defects. The remaining 34 patients had no fixed or reversible defects (nonischemic group). No patient had fixed defects only. Clinical characteristics of the two patient groups and healthy volunteers are shown in Table 1; the two patient groups were comparable in all respects except age (ischemic group mean 61.2 years, nonischemic group mean 55.9 years, p=0.025) and a trend toward more frequent history of prior myocardial infarction in the ischemic group (55% vs. 23.5% in the nonischemic group, p=0.056). No other significant differences were found in the clinical history, prior revascularization, or treatment with various commonly used medications.

TABLE 1

| | | | | P value vs. |
|---|---|---|---|---|
| Values are | Healthy | Nonischemic | Ischemic | Nonischemic |
| number | Volunteers | Group | Group | Group |
| (% of group) | (n = 21) | (n = 34) | (n = 40) | (*= < 0.05) |
| Age in years | 21.1 ± 1.2 | 55.9 ± 9.6 | 61.2 ± 10.2 | 0.025 * |
| (Mean ± SD) | | | | |
| Male | 8 (38) | 25 (73.5) | 37 (92.5) | n.s. |
| Prior MI | — | 8 (23.5) | 22 (55.0) | 0.056 * |
| Prior PTCA | — | 19 (55.9) | 24 (60.0) | n.s. |
| Prior CABG | — | 4 (11.8) | 8 (20.0) | n.s. |
| History of | — | 18 (52.9) | 22 (55.0) | n.s. |
| Hyperten. | | | | |
| History of | — | 3 (8.8) | 8 (20.0) | n.s. |
| Diabetes | | | | |
| History of | — | 2 (5.9) | 11 (27.5) | n.s. |
| Angina | | | | |
| Current smoking | — | 5 (14.7) | 4 (10.0) | n.s. |
| Former smoking | — | 10 (29.4) | 8 (20.0) | n.s. |
| History of | — | 28 (82.4) | 34 (85.0) | n.s. |
| Increase in | | | | |
| Lipids | | | | |
| Rx beta blocker | — | 24 (70.6) | 26 (65.0) | n.s. |
| Rx ACEI | — | 16 (47.1) | 18 (45.0) | n.s. |
| Rx Calcium | — | 10 (29.4) | 12 (30.0) | n.s. |
| blocker | | | | |
| Rx nitrates | — | 0 (0) | 5 (12.5) | n.s. |
| Rx ARB | — | 3 (8.8) | 2 (5.0) | n.s. |
| Rx Statin | — | 33 (97.1) | 36 (90.0) | n.s. |

SD is standard deviation; n is number; MI is myocardial infarction; PTCA is percutaneous transluminal coronary angioplasty; CABG is coronary artery bypass graft; Rx is prescription; ACEI is angiotensin converting enzyme inhibitor; ARB is angiotensin receptor blocker; and n.s. is not significant.

Analysis of exercise test data showed no significant difference between the two patient groups in maximal exercise capacity, maximal systolic blood pressure, the presence of exertional chest pain, or Duke Treadmill Score. The percentages of patients who developed ECG changes characteristic of ischemia did not differ between the two groups (nonischemic 41.2%, ischemic 37.5%, p=0.99) (Table 2). Maximal heart rate and rate-pressure product were higher in the nonischemic group and left ventricular ejection fraction was lower in the ischemic group than in the nonischemic group (51.8% vs. 57.8%, p=0.001). Table 2 summarizes the findings on exercise testing and gated imaging.

TABLE 2

| | | | | P value vs. |
|---|---|---|---|---|
| | Healthy | Nonischemic | Ischemic | nonischemic |
| | Volunteers | group | group | group |
| | (n = 21) | (n = 34) | (n = 41) | (* =< 0.05) |
| Achieved | 17.6 ± 2.8 | 10.6 ± 3.4 | 11.3 ± 3.1 | n.s. |
| Mets | | | | |
| (mean) | | | | |
| Maximal HR | 186 ± 7.5 | 142.1 ± 20.2 | 131.8 ± 18.3 | 0.026 * |
| (mean) | | | | |
| Maximal BP | 158 ± 17.8 | 179.4 ± 25.7 | 171.1 ± 22.0 | n.s. |
| (mm Hg) | | | | |
| Rate- | 21 (100) | 256 ± 56.2 | 226 ± 47.6 | 0.019 * |
| Pressure | | | | |
| Product | | | | |
| (×100) | | | | |
| Achieved ≧ | 295 ± 33.7 | 22 (64.7) | 16 (40) | n.s. |
| 85% | | | | |
| pred. max. | | | | |
| HR, no. (%) | | | | |
| Exertional | 0 | 7 (20.6) | 8 (20) | n.s. |
| chest pain, | | | | |
| no. (%) | | | | |
| Positive EKG, | 0 | 14 (41.2) | 15 (37.5) | n.s. |
| no. (%) | | | | |
| Mean | — | 57.8% | 51.8% | 0.001 * |
| Ejection | | | | |
| Fraction | | | | |
| (gated | | | | |
| SPECT) | | | | |

Met is metabolic equivalents, HR is heart rate, BP is blood pressure and SPECT is single photon emission computed tomography.

Volunteer blood was analyzed for NTproBNP only, and pre-exercise (baseline) levels were normal in all subjects. Although baseline levels of BNP and NTproBNP were normal in both ischemic and nonischemic groups, median levels were significantly higher in the ischemic group (NTproBNP 120.5 pg/mL vs. 53.5 pg/mL, p<0.0001; BNP 40.5 pg/mL vs. 16.5 pg/mL p<0.001) (Table 3). Interquartile ranges showed no overlap in NTproBNP values, and only modest overlap in BNP values. Resting NTproBNP values were lower in the healthy volunteers (median 25 pg/mL) than in the CAD patient groups (p=0.0053 vs. nonischemic group).

TABLE 3

| Values are | | | P Value | | |
|---|---|---|---|---|---|
| medians | Normal | | (vs. | Ischemic | P value |
| (interquartile | Vol. | NI group | Normal | group | (vs. NI |
| range) | (n = 21) | (n = 34) | Vol.) | (n = 40) | group) |
| Baseline | 25 | 53.5 | 0.0053 | 120.5 | <0.0001 |
| NTproBNP | (15-35) | (28-74) | | (76-158) | |
| (pg/mL) | | | | | |
| 1 minute Δ | 5 | 4 | n.s. | 14.5 | <0.0001 |
| NTproBNP | (2-9) | (0.5-9.5) | | (10.5-19.5) | |
| (pg/mL) | | | | | |
| Baseline BNP | — | 16.5 | — | 40.5 | <0.001 |
| (pg/mL) | | (9.5-30.5) | | (24-54) | |
| 1 minute Δ BNP | — | 7.5 | — | 36.5 | <0.0001 |
| (pg/mL) | | (3.5-17.5) | | (15-49.5) | |

Vol. = volunteers, NI = Nonischemic.

Both NTproBNP and BNP increased with exercise in all groups. The median incremental rise (ΔNTproBNP and ΔBNP) was almost identical in the healthy volunteers and in the nonischemic patient group (5 pg/mL vs. 4 pg/ml, p=n.s.). However, the incremental rise in the ischemic group was significantly higher than in the nonischemic group (ΔNTproBNP: 14.5 pg/mL vs. 4 pg/mL, p<0.0001; ΔBNP: 36.5 pg/mL vs. 7.5 pg/mL, p<0.0001). As with resting levels, there was no overlap in the interquartile ranges for NTproBNP and modest overlap for BNP.

Because 14 patients in the ischemic group were found to have fixed, as well as reversible defects on radionuclide images, a subset analysis was conducted on the 26 ischemic patients with reversible defects only. Results of this analysis are shown in Table 4. Median resting levels of NTproBNP and BNP for this subgroup were 118 pg/mL and 44 pg/mL, respectively, values that did not differ significantly from the values for the entire ischemic group. Similarly, median ΔNPproBNP and ΔBNP for this subgroup were 16 pg/mL and 36 pg/mL respectively; as with resting levels, the Δ values were not significantly different from the ischemic group as a whole.

TABLE 4

| | | Patients with | |
|---|---|---|---|
| | All Patients | Reversible | |
| Values are medians | with Ischemia | Defects Only | |
| (interquartile range) | (n = 40) | (n = 26) | P value |
| Baseline NTproBNP | 120.5 | 118 | n.s |
| pg/mL | (76-158) | (67.5-140.5) | |
| ΔNTproBNP | 14.5 | 16 | n.s |
| pg/mL | (10.5-19.5) | (10.5-18.5) | |
| Baseline BNP | 40.5 | 44 | n.s. |
| pg/mL | (24-54) | (25.5-52) | |
| ΔBNP | 36.5 | 36 | n.s. |
| pg/mL | (15-49.5) | (16.5-52.5) | |

The ability of baseline and Δ BNP and Δ NTproBNP levels to predict the presence or absence of ischemia in individual patients was evaluated by constructing receiver operator characteristic curves for each peptide. Table 5 shows the test characteristics at selected cut points for ΔBNP and ΔNTproBNP.

TABLE 5

| | | | | Positive | Negative |
|---|---|---|---|---|---|
| | Sensi- | | Diagnostic | Likelihood | Likelihood |
| | tivity | Specificity | Accuracy | Ratio | Ratio |
| Cutpoint Δ | | | | | |
| NTproBNP | | | | | |
| (pg/mL) | | | | | |
| 5 | 0.900 | 0.588 | 0.757 | 2.19 | 0.170 |
| 6 | 0.850 | 0.706 | 0.773 | 2.89 | 0.213 |
| 7 | 0.825 | 0.706 | 0.770 | 2.81 | 0.248 |
| 8 | 0.800 | 0.735 | 0.770 | 3.02 | 0.272 |
| Cutpoint ΔBNP | | | | | |
| (pg/mL) | | | | | |
| 9 | 0.800 | 0.559 | 0.689 | 1.81 | 0.358 |
| 10 | 0.8005 | 0.588 | 0.703 | 1.94 | 0.340 |
| 11 | 0.775 | 0.618 | 0.703 | 2.03 | 0.364 |
| 12 | 0.775 | 0.647 | 0.716 | 2.20 | 0.348 |

The area under the curve (AUC) for NTproBNP was 0.836 (95% CI 0.742-0.930), and for BNP was 0.811 (95% CI 0.713-0.908, p<0.0001 for both). Sensitivities and specificities to be higher for ΔNTproBNP than for ΔBNP at able cut points.

The correlation between induced changes in peptide levels and an estimate of the extent and severity of ischemia was assessed by comparing ΔNTproBNP and ΔBNP values to the SDS scores generated by the computer software interpretation of radionuclide images for all patients. This demonstrated a moderate positive correlation between these variables (Pearson r=0.33, p=0.004, for ΔNTproBNP, and r=0.31, p=0.007, for ΔBNP).

Analysis of the exercise ECG data showed that the sensitivity and specificity of 1 mm horizontal or downsloping ST depression for the detection of ischemia were 37.5% and 58.8%, respectively.

The ischemic and nonischemic groups were also examined by gender. The number of women in the ischemic group was too small for statistical significance (n=3), but both ΔBNP and ΔNTproBNP correctly predicted ischemia in these patients. Specificity among the nine women without ischemia was 67%. Sensitivity and specificity for men were not significantly different from the values for the original groups.

Linear binary correlation analysis found that baseline peptide levels correlated positively with age (r=0.57, p<0.0001), SSS (r=0.56, p<0.0001), SRS (r=0.45, p=0.0001), and SDS (r=0.50, p=0.0001), and negatively with maximal heart rate (r=−0.35, p=0.002) and exercise capacity (r=−0.29, p=0.01). By contrast, ΔNTproBNP and ΔBNP correlated only with SSS and SDS, and less strongly with SRS, but not with any other measured clinical or exercise-test derived variables. Logistic regression analysis showed that after correcting for other variables, ΔBNP and NTproBNP were strongly predictive of ischemia (z score 12.8, p<0.001). In a generalized linear model, Δ peptide levels accurately predicted SDS values (F ratio 10.4, p<0.001).

The results provided herein demonstrate that the measurement of pre- and post-exercise natriuretic peptides is considerably more accurate in the detection of ischemia than is ST depression on exercise electrocardiography. Comparative test characteristics of ECG findings and ΔNTproBNP and ΔBNP levels for the detection of ischemia, set at equal specificities to ECG, are shown in Table 6.

TABLE 6

| | | | Positive | Negative |
|---|---|---|---|---|
| | | | predictive | predictive |
| | Sensitivity | Specificity | value | value |
| Δ NTproBNP > | 90.0% | 58.8% | 72.0% | 83.3% |
| 5 pg/mL | | | | |
| Δ BNP > | 80.0% | 58.8% | 69.6% | 71.4% |
| 10 pg/mL | | | | |
| ≧1 mm ST | 37.5% | 58.8% | 51.7% | 44.4% |
| depression | | | | |
| on ECG | | | | |

| | Diagnostic | Positive | Negative |
|---|---|---|---|
| | accuracy | Likelihood Ratio | Livelihood Ratio |
| Δ NTproBNP > | 75.7% | 2.19 | 0.17 |
| 5 pg/mL | | | |
| Δ BNP > | 70.3% | 1.94 | 0.34 |
| 10 pg/mL | | | |
| ≧1 mm ST | 47.3% | 0.91 | 1.06 |
| depression | | | |
| on ECG | | | |

Compared to the ECG, measurement of ΔNTproBNP and ΔBNP more than doubled the sensitivity of the exercise test for ischemia (ΔNTproBNP 90%, ΔBNP 80%) with no loss of specificity. ΔNTproBNP, in particular, correctly predicted the presence or absence of ischemia almost twice as frequently as the ECG (diagnostic accuracy 75.7% vs. 47.3%).

The sensitivity of the ECG for detecting ischemia in the patients herein was in the lower range of those reported for exercise testing (Gianrossi, et al. (1989) supra), although it was similar to values reported for studies with reduced work-up bias (Froelicher, et al. (1998) supra; Morise and Diamond (1995) supra). One reason for this may be that in standard practice, exercise tests in which patients have no diagnostic changes on ECG but do not achieve 85% of predicted maximal heart rate for age are often considered indeterminate and thus censored from calculations of test accuracy; in the study provided herein such tests were considered to be negative for ischemia, since by study design it would not be know from the SPECT images whether ischemia was present or not. If ischemia was present in these cases, the ECG failed to detect it, and thus was falsely negative. Only a small number of studies have examined the ability of the exercise ECG to predict reversible defects on nuclear imaging. Two representative studies (Nallamothu, et al. (1995) J. Am. Coll. Cardiol. 25:830-6; Galassi, et al. (2000) J. Nucl. Cardiol. 7:575-83) compared EKG findings with perfusion images and found EKG sensitivities of 45.5% and 42.8%, which are similar to the findings provided herein.

Reduced regional myocardial blood flow results in a cascade of changes beginning with relaxation failure and progressing to contraction abnormalities, rise in filling pressure, ECG changes, and finally symptoms (Sigwart, et al. (1984) In: Rutishauser W, Roskamm H, eds., Silent Myocardial Ischemia pgs. 29-36). Since ECG abnormalities occur later in this process than changes in ventricular wall function, BNP would rise before ECG abnormalities appear; in other words, measuring NTproBNP or BNP rise may be more sensitive because it detects reduced myocardial blood flow at an earlier stage.

The findings provided herein indicate that in a group of patients with known coronary artery disease, measurement of plasma levels of BNP or NTproBNP before and immediately after symptom-limited exercise testing identifies patients who have inducible ischemia, defined as reversible defects on radionuclide SPECT imaging, with a high degree of accuracy. This was true whether patients were grouped by radiologist's interpretation of images or by computer software interpretation.

Accordingly, the present invention is a method for detecting cardiac ischemia in an individual. The method involves measuring the level of a natriuretic peptide in a sample isolated from an individual and comparing said level to a control, wherein an increase in the level in the sample as compared to the control is indicative of cardiac ischemia in the individual. In one embodiment of the present invention, a natriuretic peptide is brain natriuretic peptide (BNP), N-terminal probrain natriuretic peptide (NTproBNP), or a fragment thereof, e.g., a degradation product of neutral endopeptidase.

In accordance with the method of the present invention, a control can be the median level of a natriuretic peptide present in a group of patients without ischemia or, alternatively, a control can be the level of a natriuretic peptide in a first sample isolated from an individual before said individual has conducted an exercise test. Accordingly, in the latter case, the levels of a natriuretic peptide in the first sample (i.e., the control) are compared to the levels of a natriuretic peptide in a second sample isolated from the same individual after the individual has conducted an exercise test.

To measure the level of a natriuretic peptide, a sample is isolated from an individual, in general before and after an exercise test. The sample can be whole blood, plasma, urine or the like, or can be a biopsy sample, isolated according to standard clinical methods. When performed in conjunction with an exercise test, the first sample is isolated, e.g., 1 minute, 5 minutes, 15 minutes, 30 minutes or more before the exercise test and the second sample is isolated, e.g., 1 minute, 5 minutes, 15 minutes, or 30 minutes post-exercise. As BNP and NTproBNP are stable in whole blood or plasma at room temperature for 10-48 and 4-10 hours, respectively, and BNP is stable for up to 72 hours at 2-8° C., special handling of the sample is not required. Further, EDTA and protease inhibitors (e.g., aprotinin) may or may not be added to the sample after isolation to inhibit degradation.

The levels of a natriuretic peptide are measured using methods provided herein or other suitable assays such as immunoassays (e.g., RIA or EIA such as SHIONORIA BNP test (Cis Bio International, France)); noncompetitive immunoassays, or two-site (sandwich) immunometric assays using two specific monoclonal antibodies or antisera prepared against two sterically remote epitopes of the natriuretic peptide chain and the like.

It is contemplated that either an absolute increase or percent increase in the levels of a natriuretic peptide over control levels is indicative of ischemia in the individual from whom the sample was isolated. However, in a particular embodiments of the present invention, the absolute increase in sample levels over the control is used to diagnose ischemia. Various descriminative values (or cut points) for distinguishing normal from increased levels of a natriuretic peptide provide different sensitivities and specificities to the method herein. For example, a cut point yielding a high sensitivity can be used to diagnose ischemia. For NTproBNP, a cut point value for diagnosing ischemia can be in the range of 4-10 pg/mL, in the range of 4-8 pg/mL, or 5 pg/mL. For BNP, a cut point value for diagnosing ischemia can be in the range of 8-16 pg/mL, in the range of 9-12 pg/mL, or 9-10 pg/mL for BNP. In general, increasing the cut point results in a decrease in sensitivity and an increase in specificity.

It is contemplated that the method of the present invention is useful in detecting ischemia in both symptomatic and asymptomatic individuals and can also be used for prognostic purposes.

As used herein, exercise testing is defined as a cardiovascular stress test using treadmill or bicycle exercise. Alternatively, cardiovascular stress can be induced using a pharmacological agent such as dobutamine infusion. In general, the exercise testing is conducted by a skilled clinician, exercise physiologists, physician assistants, wherein the electrocardiogram (ECG), heart rate, and blood pressure of the individual being tested is monitored and recorded during each stage of exercise and during ST-segment abnormalities and chest pain. Guidelines and other considerations for standard exercise testing are well-known in the art, see, e.g., ACC/AHA 2002 Guideline Update for Exercise Testing (American College of Cardiology Foundation and the American Heart Association, Inc.). In general, either a cycle ergometer is used or a treadmill can be used according to, for example, the Bruce protocol, with 6 to 12 minutes of exercise (Myers and Froelicher (1990) Circulation 82:1839-46). Although exercise testing is commonly terminated when an individual reaches an arbitrary percentage of predicted maximum heart rate, the skilled artisan will appreciate that other end points can be used. For example, absolute indications such as a drop in systolic blood pressure of >10 mm Hg from baseline blood pressure despite an increase in workload, when accompanied by other evidence of ischemia; moderate to severe angina; increasing nervous system symptoms (e.g., ataxia, dizziness, or near-syncope); signs of poor perfusion (cyanosis or pallor); etc. Alternatively, relative indications such as a drop in systolic blood pressure of (≧10 mm Hg from baseline blood pressure despite an increase in workload, in the absence of other evidence of ischemia; ST or QRS changes such as excessive ST depression (>2 mm of horizontal or downsloping ST-segment depression) or marked axis shift; arrhythmias other than sustained ventricular tachycardia, including multifocal PVCs, triplets of PVCs, supraventricular tachycardia, heart block, or bradyarrhythmias; fatigue, shortness of breath, wheezing, leg cramps, or claudication; and the like can be used.

As the skilled artisan will further appreciate, there is a wide spectrum of values around the regression line for maximum heart rate, which can therefore be beyond the limit of some individuals and submaximal for others. The target heart rate approach has obvious additional limitations in patients receiving beta-blockers, those with heart rate impairment, and those with excessive heart rate response. Thus, the use of rating of perceived exertion scales, such as the Borg scale (Borg (1982) Med. Sci. Sports Exerc. 14:377-81) can be used in the assessment of patient fatigue.

In accordance with the method of the present invention, the levels of a natriuretic peptide before and after an exercise test can be used alone or in combination with other well-known methods for detecting cardiac ischemia. Other methods can include, e.g., stress echocardiography, electrocardiographic monitoring, blood pressure monitoring, radionuclide imaging (e.g., radionuclide angiography, myocardial perfusion imaging, or stress single-photon emission computed tomography (SPECT) myocardial perfusion imaging) and the like.

The invention is described in greater detail by the following non-limiting examples.

### EXAMPLE 1

### Methods

Seventy-four consecutive patients with documented CAD who were referred for exercise stress testing with single photon emission computed tomographic (SPECT) myocardial perfusion imaging were enrolled. Sixty-nine patients had CAD diagnosed by coronary angiography; five had prior nuclear imaging studies showing reversible defects consistent with ischemia. Patients with a history of heart failure, atrial fibrillation, pacemakers, significant valvular disease (including replacement), age >80 years, echo left ventricular ejection fraction <55%, or recent (<2 months) infarction or revascularization were excluded. Also excluded were patients taking digitalis, or whose resting ECG's showed abnormalities that would preclude interpretation of exercise-induced changes, e.g., left bundle branch block, left ventricular hypertrophy, >1 mm ST segment changes, or pre-excitation. Also enrolled were 21 healthy volunteers (mean age 21.1 years) with no history of cardiovascular disease or other significant illness.

After written informed consent, exercise testing with myocardial perfusion imaging was performed using a dual isotope, rest-stress protocol. Four mCi ²⁰¹thallous chloride were injected and resting images acquired using a Philips (Cleveland, Ohio) IRIX™ three-headed gamma camera. Patients then underwent symptom-limited exercise testing on a treadmill using a Bruce protocol. Exercise was terminated for fatigue, marked dyspnea, exercise-limiting angina, >20 mmHg decrease in systolic BP, or >3 mm ST depression. No cases of serious arrhythmia or severe hypertension necessitating termination of exercise were observed. Ninety seconds prior to the termination of exercise, 33 mCi of ^{99m}technetium tetrofosmin (Amersham Healthcare, Arlington Heights, Ill.) were administered and stress images were subsequently acquired with ECG gating. Healthy volunteers underwent symptom-limited exercise testing without myocardial perfusion imaging.

Prior to exercise, after 10 minutes supine rest, and again at one minute post-exercise, a venous blood sample was collected via an indwelling 20 gauge IV cannula. Samples were placed in EDTA anticoagulated polyethylene tubes and the plasma separated, aliquoted, and frozen at −80° C. until analysis.

Exercise electrocardiograms were interpreted by an experienced physician blinded to the interpretation of perfusion images and results of analysis of blood samples. ECG's were interpreted as positive for ischemia if they showed ≧1 mm horizontal or downsloping ST depression at 0.80 milliseconds after the J-point during exercise or recovery. ECG's showing no significant ST depression at peak exercise were interpreted as negative for ischemia at that level of exercise regardless of the maximal heart rate achieved.

Radionuclide SPECT images were interpreted by an experienced radiologist, blinded to clinical history, exercise test data, and the results of analysis of blood samples. Images were classified as having no perfusion defects, fixed defects only, fixed and reversible defects, or reversible defects only; the defects were also characterized by size, severity, and vascular territory. Images were also assessed independently of the radiologist's interpretation with a computer software program (QPS, Cedars Sinai, Los Angeles), using a 20 segment polar model which compares acquired photon counts in each segment to a gender-specific database of normal studies. Values from 0 to 4 were assigned to each segment, 0 being normal and 4 being no counts; the total was expressed as a summed stress score (SSS), a summed rest score (SRS), and a summed difference score (SDS), the latter indicating the degree of reversibility. Myocardial function was assessed using quantitated gated SPECT imaging.

Resting and post-exercise blood samples were analyzed in batches for NTproBNP, using an electrochemiluminescent immunoassay (Roche Diagnostics, Indianapolis, Ind.) on an ELECSYS® 1010 autoanalyzer, and for BNP using a fluorescent point-of-care immunoassay (BIOSITE®, San Diego). Coefficients of variation for the assays were: NTproBNP 2.9-6.1% and BNP 9.9-12.5% (Yeo, et al. (2003) Clin. Chim. Acta 338:107-115). NTproBNP assays were run in duplicate.

SPSS, MICROSOFT® EXCEL®, and ANALYSE-IT™ statistical software were used in our analysis. Student's t-test and Mann Whitney modified student's t-test were used to compare means and medians, respectively, of continuous variables; chi square was used to compare dichotomous variables. All tests were two-tailed and corrected for multiple comparisons. Logistic regression and linear binary correlations were performed with SPSS.

What is claimed is:

1. A method for detecting cardiac ischemia in an individual comprising measuring the level of a natriuretic peptide in a sample isolated from an individual and comparing said level to a control, wherein an increase in the level in the sample as compared to the control is indicative of cardiac ischemia in the individual.

2. The method of claim 1, wherein the natriuretic peptide is brain natriuretic peptide, N-terminal probrain natriuretic peptide, or a fragment thereof.

3. The method of claim 1, wherein the control is isolated from an individual before the individual has conducted an exercise test and the sample is isolated from the same individual after the individual has conducted an exercise test.
